(19) United States
(12) Plant Patent Application Publication
(10) Pub. No.: US 2008/0282436 P1
(43) Pub. Date: Nov. 13, 2008

(54) Zoysiagrass plant "TMX"

(75) Inventor: Kunio Matsui, Nishikamo-gun (JP)

Correspondence Address: OBLON, SPIVAK, MCCLELLAND MAIER & NEUSTADT, P.C., 1940 DUKE STREET, ALEXANDRIA, VA, 22314, US

(73) Assignee: TOYOTA JIDOSHA KABUSHIKI KAISHA, Toyota-shi (JP)

(21) Appl. No.: 12/149,800

(22) Filed: May 8, 2008

(30) Foreign Application Priority Data
May 8, 2007 (JP) 21038

(51) Int. Cl. A01H 5/00

(52) U.S. Cl. PLT/390

(57) ABSTRACT

The present culivar TMX (Zoysia matrella Merr.) advantageously has a very slow growing rate in terms of plant height as compared with existing cultivars and requires mowing not more than once a year so as to be maintained in fair condition and allows the amount of fertilizer to be reduced. It also has a high-level of shade tolerance.

### LATIN NAME OF THE GENUS AND SPECIES OF THE PLANT CLAIMED

The present invention relates to the species Zoysia matrella Merr.

### VARIETY DENOMINATION

“TMX.”

### BACKGROUND OF THE INVENTION

### Field of Invention

The present invention relates to a new and distinct perennial variety of Zoysiagrass asexually reproduced and selected.

### SUMMARY OF THE INVENTION

The present invention relates to a new and distinct Zoysiagrass cultivar (Zoysia matrella Merr.) named “TMX.”

Zoysiagrass is a popular lawn grass which has been widely used as greening turf. The turf thereof, however, should be mown 3 to 5 times per year so as to be maintained in beautiful condition. Since the newly developed TMX advantageously has a very slow growing rate in terms of plant height as compared with conventional cultivars, mowing is required not more than once a year to maintain fair condition and the amount of fertilizer can be reduced to almost less than half. Therefore, labors and cost necessary for maintenance of the turf as well as clipping from lawn-mowing can be reduced. Furthermore, the cultivar can be readily grown in a place where a lawnmower cannot be conveniently used, such as on a rooftop of a building. In addition, TMX has superior shade tolerance to that of a conventional cultivar, and thus can be raised even in a place with insufficient sunlight exposure.

In addition, it is expected that prevalence of TMX facilitates greening of the rooftop and schoolyard and leads to alleviation of heat island phenomenon and reduction of the amount of CO₂.

For purposes of registration under the “International Convention for the Protection of New Varieties of Plants” (generally known by its French acronym as the UPOV Convention) and noting sections 1612 of the Manual of Patent Examining Procedure, it is proposed that the new variety of Zoysiagrass of the present invention be named “TMX.”

### BRIEF DESCRIPTIONS OF THE ILLUSTRATIONS

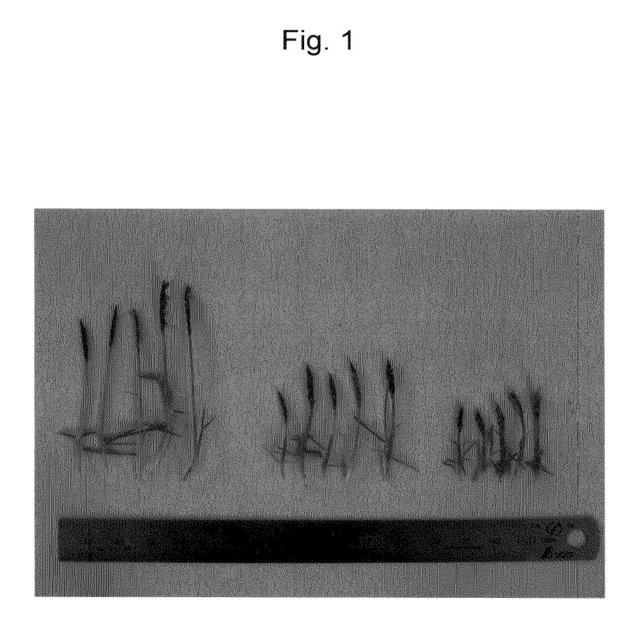

FIG. 1 is a set of photographs comparing spikestems of control cultivars and the present cultivar (TMX) (Left: Emerald; Middle: TM9; Right: TMX). Photographed on May 22, 2006 in Nishikamo-gun, Aichi, Japan.

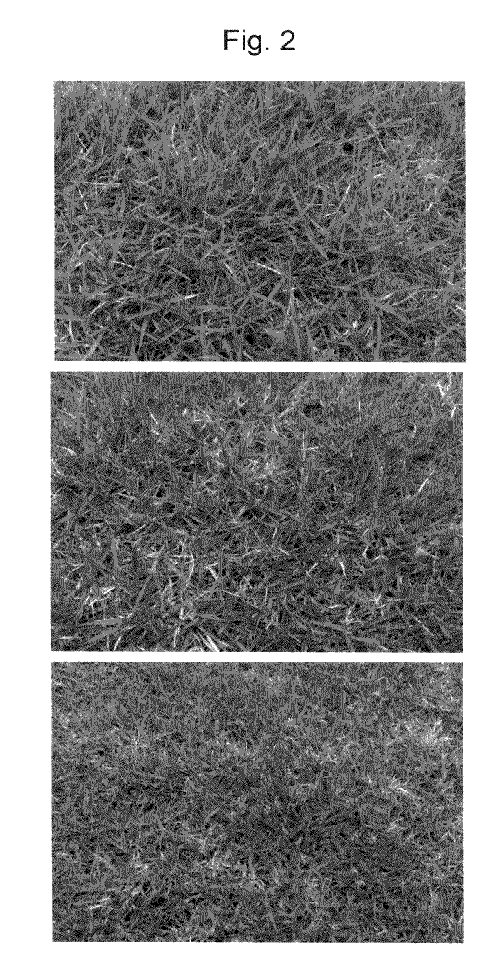

FIG. 2 is a set of photographs comparing grass shapes of control cultivars and the present cultivar (TMX) (Upper: Emerald; Middle: TM9; Lower: TMX). Photographed on May 12, 2006 in Nishikamo-gun, Aichi, Japan.

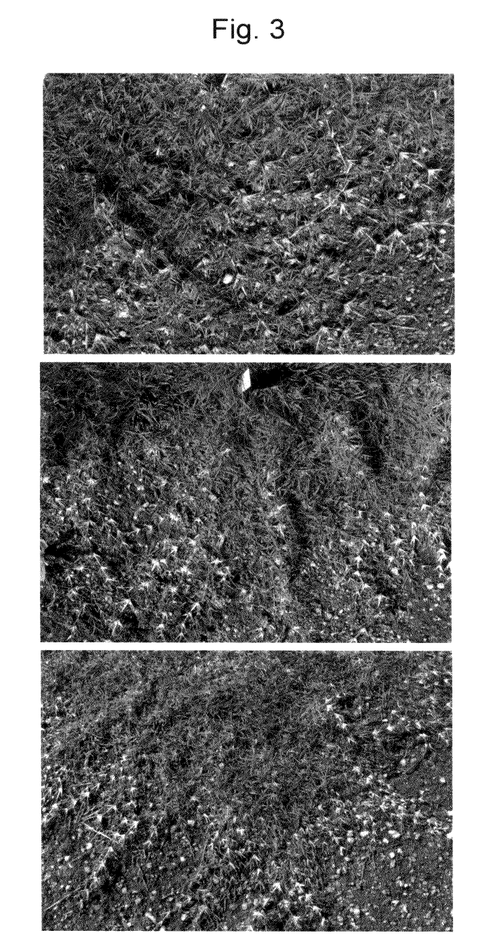

FIG. 3 is a set of photographs comparing stolons of control cultivars and the present cultivar (TMX) (Upper: Emerald; Middle: TM9; Lower: TMX). Photographed on October 20, 2005 in Nishikamo-gun, Aichi, Japan.

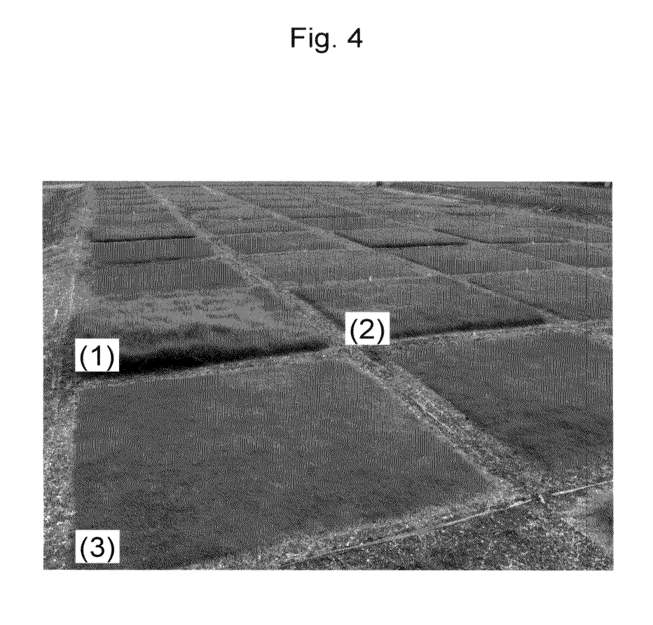

FIG. 4 is a photograph showing a full view of test field for the present cultivar (TMX) ((1): Individually planted plot of Emerald; (2): Individually planted plot of TM9; (3); Individually planted plot of TMX). Photographed on September 25, 2006 in Nishikamo-gun, Aichi, Japan.

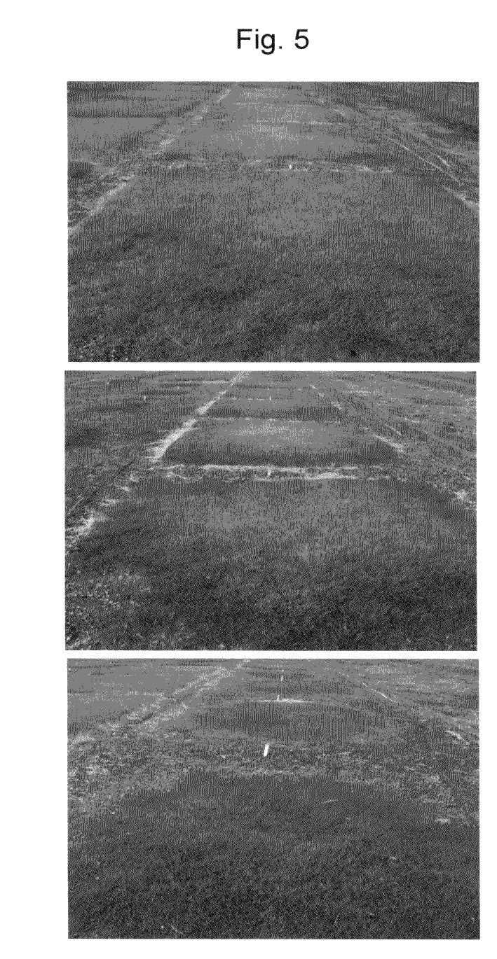

FIG. 5 is a set of photographs comparing control cultivars and the present cultivar (TMX) in terms of plant vigor in fall (Upper: Emerald; Middle: TM9; Lower: TMX). Photographed on November 13, 2006 in Nishikamo-gun, Aichi, Japan.

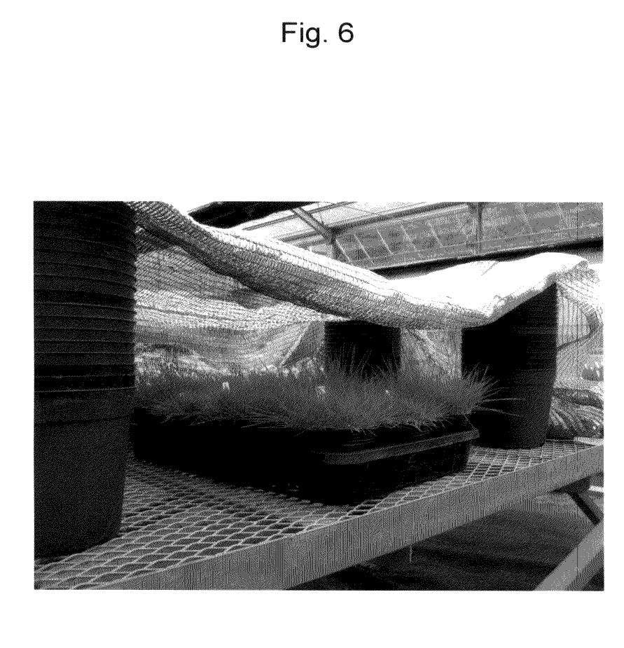

FIG. 6 is a photograph showing culture of control cultivars and the present cultivar (TMX) in a shade tolerance test with the use of a light-shading net. Photographed on September 7, 2006 in Nishikamo-gun, Aichi, Japan.

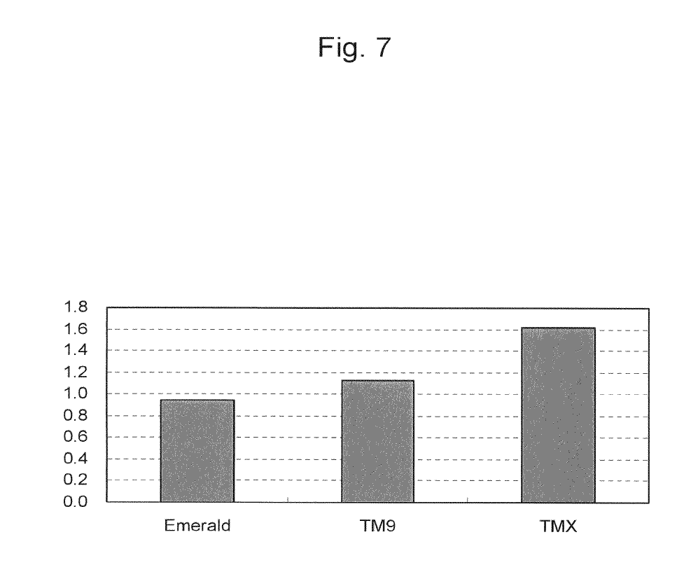

FIG. 7 is a graph showing the relative amounts of growth with 75% light shielding in a shade tolerance test (light shielding/untreated plots).

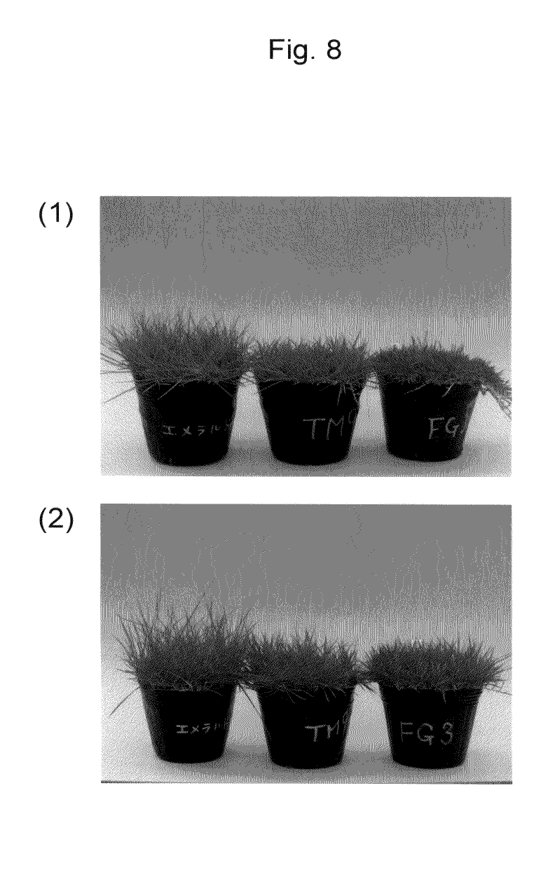

FIG. 8 is a set of photographs comparing grass shapes after culture in a shade tolerance test (Panel (1): untreated plots; Panel (2): light shielding plots) (Left: Emerald; Middle: TM9; Right: TMX in Panels (1) and (2)). Photographed on September 7, 2006 in Nishikamo-gun, Aichi, Japan.

### DETAILED BOTANICAL DESCRIPTION OF THE PLANT

1. Characteristics of the Plant Body of the Present Invention

1) Overview

Seeds (50,000 seeds) of a lawn cultivar “TM9” (Zoysia matrella Merr.) were seeded (June 2003), 70 individuals exhibiting short plant height as pot seedlings were selected from the germinated individuals (in the year 2003), and 6 individuals were selected from among these seedlings based on their growing properties as a pot seedling (in the year 2004). They were planted in a field and an outdoor cultivation test was initiated (in the year 2005). The present cultivar was obtained after completion of cultivation tests in various locations throughout Japan (completed in the year 2006). Characteristics of the present cultivar are as follows: extremely creeping plant form; spikestem of medium-sized thickness and short length; dense stolons of medium-sized thickness; leaves of very short length, very narrow leaf width, and a mild leaf color; short spike length and thick spike color; medium spikelet length, narrow spikelet width, and a small number of spikelets; moderate initial growth; moderate plant vigor in spring and good plant vigor in fall; medium (not early nor late) timing in spike-sprouting; spike-sprouting only in spring with a medium number of spikes; medium (not early nor late) timing in turning green; medium (not early nor late) timing in turning red; medium winter hardiness; good summer tolerance; strong shade tolerance; medium drought tolerance; medium salinity tolerance; and medium tread pressure resistance.

2) Comparison with Control Cultivars (Table 1)

TABLE 1

| | | | Present | | |
|---|---|---|---|---|---|
| | | | cultivar | Control cultivar | |
| | Item | Trait | TMX | Emerald | TM9 |
| 1 | Height | Height of | Low | High | Middle |
| | | mature plant | (2.0 cm) | (14.0 cm) | (5.7 cm) |
| 2 | Plant | Plant | Extremely | Intermediate | Creeping |
| | | | creeping | | |
| 3 | Stem | Thickness of | Medium | Medium | Medium |
| | | spikestem | (0.7 mm) | (0.7 mm) | (0.7 mm) |
| | | Length of | Short (2 cm) | Medium | Short (2 cm) |
| | | spikestem | | (5 cm) | |
| 4 | Stolon | Density of | Dense | Dense | Extremely |
| | | stolons | | | dense |
| | | Thickness of | Medium | Medium | Medium |
| | | stolon | (1.0 mm) | (1.1 mm) | (1.1 mm) |
| 5 | Foliage | Leaf length | Extremely | Short (8 cm) | Extremely |
| | | | short | | short to short |
| | | | (1 cm) | | (4 cm) |
| | | Leaf width | Extremely | Narrow | Narrow |
| | | | narrow | (1.6 mm) | (1.8 mm) |
| | | | (1.2 mm) | | |
| | | Leaf color | 141C: Strong | 144A: | 137A: |
| | | | yellowish | Yellow | green group |
| | | | green group | green group | |
| 6 | Spike | Spike length | Short | Short | Short |
| | | | (12 mm) | (17 mm) | (14 mm) |
| | | Spike color | 183A: | 183A: | 183A: |
| | | | Grayed | Grayed | Grayed |
| | | | purple group | purple group | purple group |
| | | Spikelet | Medium | Medium | Medium |
| | | length | (2.7 mm) | (3.2 mm) | (2.8 mm) |
| | | Spikelet | Narrow | Medium | Medium |
| | | width | (0.8 mm) | (1.2 mm) | (1.1 mm) |
| | | Number of | Few (17) | Few (16) | Few (17) |
| | | spikelet | | | |
| 7 | Initial | Initial | Medium | Medium | Medium |
| | growth | growth | | | |
| 8 | Plant | Plant vigor | Moderate | Moderate | Moderate |
| | vigor | in spring | | | |
| | | Plant vigor | Good | Good | Good |
| | | in fall | | | |
| 9 | Spike- | Beginning of | Medium | Medium | Medium |
| | sprouting | spike- | (not early | (not early | (not early |
| | time | sprouting | nor late) | nor late) | nor late) |
| 10 | Spike- | Spike- | Spike- | Spike- | Spike- |
| | sprouting | sprouting | sprouting | sprouting | sprouting |
| | properties | in spring/fall | only | only | only |
| | | | in spring | in spring | in spring |
| | | Number of | Medium | Medium | Many |
| | | spikes | | | |
| 11 | Growing | Timing to | Medium | Medium | Medium |
| | period | turn green | (not early | (not early | (not early |
| | | | nor late) | nor late) | nor late) |
| | | Timing to | Medium | Medium | Medium |
| | | turn red | (not early | (not early | (not early |
| | | | nor late) | nor late) | nor late) |
| 12 | Winter | Good/Bad in | Medium | Medium | Medium |
| | hardiness | winter | | | |
| | | hardiness | | | |
| 13 | Summer | Good/Bad in | Good | Good | Good |
| | tolerance | summer | | | |
| | | tolerance | | | |
| 14 | Environ- | Shade | Strong | Medium | Medium |
| | mental | tolerance | Medium | Medium | Medium |
| | tolerance | Drought | | | |
| | | tolerance | | | |
| | | Salinity | Medium | Medium | Medium |
| | | tolerance | | | |
| 15 | Tread | Tread | Medium | Medium | Medium |
| | pressure | pressure | | | |
| | resistance | resistance | | | |

Colors are based on the RHS color chart.

3) Other Characteristics of the Plant Body of the Present Invention

Length of internode: 18 mm

Diameter of internode: 1.0 mm

Blade villus: Hairs absent on adaxial and abaxial leaf surface

Ligule hair: 0.1 mm in length, continuously

Color of anther: 183D grayed purple group

Color of stigma: 8C reverse yellow group

Average number of florets per raceme: 17

Total length of culm up to the node immediately below the flag leaf (including the flower portion): 1.4 cm

Length of stalk of inflorescence: 0.7 cm

Length of flower portion: 1.2 cm

Color of mature spikelet: 161D grayed yellow green group

Color of stolon: 60B red purple group and 144D yellow group
4) Sensitivity to Diseases or Insect Pests

TMX did not exhibit sensitivity to diseases or insect pests that are generally found in plants of the Zoysia genus over 21 months starting from April in 2005 in Japan.
* Notes:

Type of plant: lawn

Name of the applied cultivar: TMX

Cultivator: Kunio Matsui

Address of the applicant: 1 Toyota-Cho, Toyota-shi, Aichi, Japan

Cultivation site: Nishikamo-gun, Toyota-shi, Aichi, Japan (Latitude: N 35° 08′ 06″; Longitude: E 137° 05′ 59″)

Place where characteristics research was conducted: Nishikamo-gun, Toyota-shi, Aichi, Japan (Latitude: N 35° 08′ 06″; Longitude: E 137° 05′ 59″)

Name of researcher who conducted characteristics research: Kunio Matsui

Period when characteristics research was conducted: 2005-2006

Control cultivar (most approximate cultivar): TM9
5) Characteristics by which the Present Cultivar is Distinguished from the Control Cultivars
a) Control cultivars

Emerald

TM9
b) Distinguishing characteristics

The present cultivar can be distinguished from Emerald in that the present cultivar has a low height (2.0 cm) (whereas Emerald has a high height (14.0 cm)); an extremely creeping plant form (whereas Emerald has an intermediate plant form); short-length spikestems (2 cm) (whereas Emerald has medium-length spikestems (5 cm)); very short leaves (1 cm) (whereas Emerald has short leaves (8 cm)); an extremely narrow leaf width (1.2 mm) (whereas Emerald has a narrow leaf width (1.6 mm)); a strong yellowish green leaf color (141C) (whereas Emerald has a yellow green leaf color (144A)); a narrow spikelet width (0.8 mm) (whereas Emerald has a medium spikelet width (1.2 mm)); and a strong shade tolerance (whereas Emerald has a medium shade tolerance).

The present cultivar can be distinguished from TM9 in that the present cultivar has a low height (2.0 cm) (whereas TM9 has a medium height (5.7 cm)); an extremely creeping plant form (whereas TM9 has a creeping plant form); dense stolons (whereas TM9 has extremely dense stolons); very short leaves (1 cm) (whereas TM9 has short to extremely short leaves (4 cm)); an extremely narrow leaf width (1.2 mm) (whereas TM9 has a narrow leaf width (1.8 mm));a strong yellowish green leaf color (141C) (whereas TM9 has a green leaf color (137A)); a narrow spikelet width (0.8 mm) (whereas TM9 has a medium spikelet width (1.1 mm)); a medium number of spikes (whereas TM9 has a large number of spikes); and a strong shade tolerance (whereas TM9 has a moderate shade tolerance).
6) Cultivation Conditions for Characteristic Research Experiment of Above 1) to 5)
a Cultivation site: Nishikamo-gun, Aichi, Japan (Latitude: N 35° 08′ 06″; Longitude: E 137° 05′ 59″)
b Cultivation time and period: 21 months from April, 2005
c Cultivation method (Examples: cultivation style such as on bare ground, in a facility, field planting or pot planting, cultivation scale, etc.)

Pot seedlings reproduced by cutting rootstocks and stolons and planting them in soil were raised in greenhouse for two months and fix planted on bare ground in April along with control cultivars in order to perform a characteristic research experiment. Five individuals (triplicates) were fix planted at a density of one seedling per an area of 200 cm×200 cm in an individually planted plot while they were fix planted in triplicates at a density of one seedling per an area of 15 cm×15 cm in a dense plated test plot having an area of 150 cm×150 cm thereby performing a characteristic research experiment.
7) Shade Tolerance Test
a) Test method

Three pot seedlings (pot size: φ 12 cm) of each of Emerald, TM9, and TMX were raised under 75% light shielding with a light-shading net for approximately 2 months from July 6 to September 7 in 2007 in Nishikamo-gun, Aichi, Japan (Latitude: N 35° 08′ 06″; Longitude: E 137° 05′ 59″) (FIG. 6). In addition, three pot seedlings of each of the above cultivars were raised under conditions without the use of a light-shielding net (untreated plot). The aerial portions of each cultivar were mowed. Then, the mean value of the weights of the mowed parts of three pot seedlings that had been raised while covered with a light-shading net was divided by a mean value of the weights of the mowed parts of three pot seedlings in the untreated plot. Thus, the relative amount of growth was calculated.
b) Results

The seedlings of Emerald and TM9 that had been raised with light shielding grew to an extent comparable to that of those in the untreated plot. Meanwhile, the amount of growth of the seedlings of TMX that had been raised with light shielding was approximately 1.6 times greater than that of those in the untreated plot (FIG. 7). In addition, the leaf length became longer in the case of every cultivar raised with light shielding. However, no abnormalities in leaves or the like were found (FIG. 8).

Accordingly, the shade tolerance of TMX was likely to be stronger than that of Emerald and of TM9.

2. Reproduction Method

Vegetative propagation: rootstocks, stolons, tillers, and turf

3. Conditions of Keeping and Storage the Plant having Identifiable Characteristics

Place where the plant is maintained and/or stored: Nishikamo-gun, Aichi, Japan (Latitude: N 35° 08′ 06″; Longitude: E 137° 05′ 59″)

Method of maintenance/storage: pot planting and ground planting

4. History of Cultivating the Present Cultivar

1) Material of New Cultivar

Mother: TM9 (Zoysia matrella Merr.)

Father

Family tree
2) Cultivation Site

Nishikamo-gun in Aichi, Japan (Latitude: N 35° 08′ 06″; Longitude: E 137° 05′ 59″)
3) History of Cultivating the Cultivar

Self-fertile seeds of TM9 were seeded in June, 2003, and individuals exhibiting short plant height as pot seedlings were selected from the germinated individuals. The thus selected lines were reproduced by root separation in April 2004 and the uniformity of these vegetative propagated lines was respectively confirmed in greenhouse by January, 2005. Newly root separated lines (January 2005) were fix planted in a field in April 2005. A line exhibiting a short leaf length and a satisfactory initial growth was selected and the stability thereof was confirmed to complete the cultivating of the new cultivar on December 28, 2006.

5. Main Use of the Present Cultivar

The present cultivar can be used for ground covering of a park, garden, etc.

6. Other Items in Relation to the Cultivation of the Present Cultivar

1) Applicable Area

Warm area
2) Specific Cultivation Site in Japan
Address (Zip code: 470-0201):

1099 Kurozasa-Marune, Miyoshi-machi, Nishikamo-gun, Aichi, Japan (Latitude: N 35° 08′ 06″; Longitude: E 137° 05′ 59″)

Facility for travel: Meitetsu Toyoda line (Closest station: Miyoshigaoka)
3) Cultivation Style: Normal Cultivation. Bare Ground.
Seasons suitable for seeding, planting, etc.

Other reproduction method: Vegetative propagation by stolons from the beginning of April to the beginning of May/year round
Blooming season, harvest season, and any other seasons in cultivation stage suitable for specifying characteristics of the present cultivar:

Blooming season: from the beginning to the end of May /year round

Greening season: from the end of April to the end of October/year round
4) Other Items to be Mentioned for the Cultivation of the Present Cultivar

The present cultivar is extremely creeping in plant form and short in leaf length, which allows weeds readily to grow. Frequent weeding is therefore required.
5) Information about the Cultivation Technique and Raising Condition of the Present Cultivar is Available from the Below:
Address (Zip code: 470-0201):

1099 Kurozasa-Marune, Miyoshi-machi, Nishikamo-gun, Aichi, Japan

Name: Kunio Matsui (TEL 81-0561-36-8441)

7. Other Information

When the present cultivar is allowed freely to grow, the plant height (i.e. the height from the ground to the tip of the leaf blade) is as about ¼ of that of Emerald and half of that of TM9.

What is claimed is:

1. A new and distinctive variety of Zoysia matrella Merr. plant named TMX as illustrated and described in the present specification.
